(19) United States
(12) Patent Application Publication
(10) Pub. No.: US 2025/0281086 A1
(43) Pub. Date: Sep. 11, 2025

(54) System And Method For Behavioral Health Therapy Tool

(71) Applicant: Tamara Allen Bush, Keller, TX (US)

(72) Inventor: Tamara Allen Bush, Keller, TX (US)

(21) Appl. No.: 19/062,199

(22) Filed: Feb. 25, 2025

Related U.S. Application Data
(60) Provisional application No. 63/561,819, filed on Mar. 6, 2024.

(51) Int. Cl. A61B 5/16; A61B 5/00; A61B 5/0205; G16H 10/60

(52) U.S. Cl. CPC A61B 5/165; A61B 5/0205; A61B 5/725; A61B 5/742; G16H 10/60; A61B 5/4836

(57) ABSTRACT

A system for online behavior modification of a user having an internet-connected device, a content monitor recording content identifiers of internet content accessed under the control of the user, at least one biometric sensor of the user concurrent with accessing the internet content, a server receiving content identifiers conditions associated with the user, an arousal detection filter configured to determine a level of arousal of the user associated with accessing internet content identified by the content identifiers, and an activity logger configured to record into a user activity log stored in computer-readable memory the declared level of arousal and the identifiers.

### BENEFIT CLAIM TO FILING DATE(S) OF EARLIER FILED PATENT APPLICATION(S)

This patent application claims benefit of the filing date of U.S. Provisional Patent Application 63/561,819, our docket FGP23TAB1P, filed on Mar. 6, 2024, by Tamara Allen Bush.

### FIELD OF THE INVENTION

This invention relates to electronic, optical, biomedical, and computer-based systems and methods for assisting a mental health therapist in delivering behavioral modification therapies.

### BACKGROUND OF THE INVENTION

A large percentage of the world's population are incapable of self-imposed limits on their internet use and arousal to its content. Digital markets around the world include an estimated 8 billion people with internet addiction, and of that number, 2.6 billion have problematic online behavior with cellphones, 1.5 with internet, and 1.4 with social media according to a large 2021 study by Mediascope.

Per the International Statistical Classification of Diseases and Related Health Problems (ICD-10), the most cited physical problems from internet use include loss of sleep, visual issues, muscular/skeletal issues, obesity, carpal tunnel syndrome and seizures. Mental health problems include an inability to fulfill obligations, continuation and escalation of use despite the consequences, losses, withdrawal, failed efforts to stop, loss of control, loss of time, preoccupation, distracted driving distracted driving causing fatalities, depression, anxiety, attention problems and compulsivity.

Further, problematic internet behaviors can leave so-called digital footprints, which are permanent records that define individuals' identities and limit future opportunities based on mistakes they have made in the past.

The combination of the physical and mental issues of internet addiction can devastate or end the lives of the individual suffering from the affliction as well as that of the people around them.

### SUMMARY OF THE EXEMPLARY EMBODIMENTS OF THE INVENTION

A system is disclosed and illustrated using one or more exemplary embodiments for online behavior modification of a user having an internet-connected device, a content monitor recording content identifiers of internet content accessed under the control of the user, at least one biometric sensor of the user concurrent with accessing the internet content, a server receiving content identifiers conditions associated with the user, an arousal detection filter configured to determine a level of arousal of the user associated with accessing internet content identified by the content identifiers, and an activity logger configured to record into a user activity log stored in computer-readable memory the declared level of arousal and the identifiers.

In some embodiments of the present invention, a dashboard is configured to retrieve the activity log and to display one or more records in a user-readable format on a computer user interface.

In some embodiments of the present invention, the dashboard is further configured to receive from an administrator one or more inputs to review, modify and save one or more user-specific criteria, and wherein the arousal detection filter is further configured to use the user-specific criteria in the declaring of the level of arousal of the user.

In some embodiments of the present invention, the at least one internet-connected device comprises one or more devices is selected from the list consisting of a smart watch, a smart phone, a game system, a tablet computer, and a personal computer.

In some embodiments of the present invention, the at least one biometric sensor comprises one or more sensors is selected from the list consisting of photoplethysmography (PPG) sensor, remote photoplethysmography (rPPG) sensor, respiratory rate (RR) sensor, heart rate (HR) sensor, blood oxygen saturation (SpO2) sensor, blood pressure (BP) sensor, and galvanic skin response (GSR) sensor.

In some embodiments of the present invention, the one or more identifiers of internet content is selected from the list consisting of a web page type, a streaming audio type, a streaming video type, a movie rating type, a Universal Resource Locator (URL), an internet protocol (IP) address, a removable memory type, a non-removable memory type, a data connection encryption type, and a virtual private network (VPN) type.

In some embodiments of the present invention, the user activity log is stored in a database computer-readable memory structure. In some embodiments of the present invention, the user activity log is stored in an encrypted computer-readable memory structure.

### BRIEF DESCRIPTION OF DRAWINGS

The figures presented herein, when considered in light of this description, form a complete disclosure of one or more embodiments of the invention, wherein like reference numbers in the figures represent similar or same elements or steps.

FIG. 1 illustrates an architecture according to at least one embodiment of the present invention is shown.

FIG. 2 sets forth a computing platform suitable for improvement according to the methods, processes and other elements of the present invention.

### DETAILED DESCRIPTION

The present inventor has realized that internet users need a way to protect their digital footprints or, if their footprint is compromised, they need a way to empirically show positive change to employers, family, community or legal entities. Because internet addiction is still poorly understood, there continues to be skepticism of its existence even among experts in addiction research because there are no physiological changes that accompany excessive internet use like there are with other types of substance use disorders. The physiological changes affect the brain where it is harder to detect. Research in 2011 by Hilton cites addiction disorders are labeled by neurologists as hypofrontal syndromes resulting in damage to the “breaking system” of the brain. They label two types of addiction disorders, endogenous where the user manipulates the dopamine production of the body through stimulus and exogenous where the user manipulates dopamine production using a substance. Research in 2015 shows that excessive internet use exhibits biological changes in the prefrontal cortex that mirror changes seen in other addiction syndromes. Neuroimaging shows changes in brain structure occurring in the temporal cortex and ventral striatum associated with compromised executive function and increased impulse risk occurs with uncontrolled internet use (Zhu, Yunqi & Zhang, Hong & Tian, Mei).

Medication management with cognitive behavioral therapy are the traditional means of outpatient treatment for internet addiction. Higher treatment levels of care are both expensive and temporary. These modalities are somewhat effective but do not eliminate the actual problem because they fail to address human executive function when the user is online. It is not realistic for persons to abstain from online use because much of daily function involves being online.

Blocking software installed on a user's internet-connected device can be easily disabled by the user. And, while biometric devices can measure telemetry of the user, measurement alone will not result in a behavioral change for online user.

As such, the present inventor has realized that there is an unmet need in the art to provide technology that not only detects, records and reports addictive behaviors during online use (browsing, games, searching, etc.), this new technology must also enable actionable therapies that will lead to modification of the behavior of the user in improve mental health outcomes.

The novel behavioral health therapy tool of the present invention can be used by therapists and patients to significantly reduce problematic internet behaviors and improve online behaviors. The tool, in at least one embodiment, is compliant with local, regional and national privacy rules, such as Health Insurance Portability and Accountability Act of 1996 (“HIPPA”) in the United States. Embodiments may combine technology and biometrics to be leveraged by a therapist to help persons struggling with problematic internet behavior to extinguish these negative behaviors.

In at least one embodiment, the behavioral health tool is issued to patients by their therapist along with a treatment plan. The tool may be provided as a software service (SaaS) and may include, in some embodiments, a wearable biometric monitoring system that tracks and measures the patient's biometric fingerprint and arousal levels. This monitoring data is synchronized with computer-based processes that monitor their internet activity to warn of problematic areas (content sources, content types, etc.) online.

The computer-based processes according to the present invention combines this information to provide a range of healthy versus unhealthy arousal levels on a user interface dashboard that can be used by the therapist and patient to provide effective intervention and to digitally track the patient's behavioral improvement. Through a combination of the tracked activity and the therapy plan administered by a therapist or other mental health professions, a patient is enabled to understand their arousal template and indicators of their own biometrics that will serve as a warning to protect against future problematic behavior.

As such, use of embodiments of the invention may decrease problematic internet behavior and increase in the patient's self esteem, mental, and physical health, resulting in better executive functioning when online. Auxiliary problems and costs of insomnia, vision loss, muscular skeletal issues, divorce, uncontrolled spending, job losses and other risks may also decrease. The associated cost savings for individuals, communities, businesses, and healthcare align with national mental health authorities' mission to improve the health of all people and further clinical and translational research to better address the problem of internet addiction and the associated relational and economic costs.

In some embodiments of the present invention, the system provides an extensible platform which not only leverages currently-available biometric arousal measurement technology, but also can incorporate future biometric arousal measurement technology to extend the capabilities, accuracy and coverage of the tool. In this manner, the tool and the platform it provides may provide the patients and clients additional resiliency safe guards to use their pre-frontal cortex/executive functions with improved cognitive ability, decreased impulsive decision-making, and increased impulse control. Going beyond simple blocking software which does not assist the user in retraining their brain, and going beyond medications which are only effective to the extent the user complies with the treatment program, the embodiments of the present invention will assist the user in actually changing their brain activities to decrease the impulsive and/or addictive behavior.

A First Embodiment. Because this present invention represents a very early stage of transitioning state-of-the-art biometric measurement technology into practical and operational technology for broad use, development and testing is broken disclosed in two example embodiments. In a first example embodiment, one or more sensors including variable heart rhythm (VHR), blood pressure (BP), galvanic skin response (GSR) and respiration rate (RR), are used to detect and track arousal level of the user and to correlate this time-tagged information to the user's online activities, such as web pages being visited (URL's, IP addresses, etc.), type of content being accessed (web pages, videos, chats, private messaging, etc.), type of connection being used (encrypted protocols, obscured ID, alternate ID, VPN, etc.), time of day, day of week, etc. Each of these sensors and web activity tracking technologies are readily available today, and will be integrated through custom processes being executed by the user's web-connected device (smart phone, computer, tablet, smart TV, gaming console, in-vehicle browser, etc.) and on one or more remote server computers. A therapist, with appropriate online credentials to protect patient privacy, may utilize a dashboard via the remote server(s) to set arousal thresholds for sending alerts, review arousal history correlated to browsing and online content access history, and provide the user with appropriate behavioral modification therapy leveraging the information.

In at least one embodiment, the system may suggest to the therapy a sequenced set of steps or stages in which the user's allowed arousal level before alerting is gradually reduced over time, giving the user time and opportunity to learn the new behavioral techniques, and to commit them to habit, thereby instilling confidence in themselves that they can overcome their current challenges. This confidence will, in turn, encourage continued compliance and continued effort with the behavioral modification program of therapy.

This first example embodiment may use one or more of the typical sensors present in smart phones, game consoles, tablet computers, and desktop computers, as available, including but not limited to accelerometer, gyroscope, magnetometer, proximity sensor, GPS, network communications listener, wi-fi listener, NFC listener, and bluetooth listeners. Not all devices will allow all functions, or in some devices, certain trusted permissions must be obtained by the technology supplier for access to the sensors (and listeners).

In this first example embodiment, a signal or pattern processor, including a rules base, if required, receives the one or more sensor and/or listener streams of data, and determines if the user is in a state of low, safe, or high arousal. In some embodiments, this may be determined by an absolute threshold, such as a certain blood pressure level or certain heart rate. In other embodiments, this may be done using an adaptive or context-sensitive process, such as rate of increase of heart rate, or a heart rate threshold keyed to the content being consumed (e.g., sports broadcasts may be allowed a higher HR threshold to be declared “high arousal”, whereas other video content may be allowed a lower HR threshold to be declared “high arousal”, etc.).

A Second Embodiment. In a second example embodiment, very newly-available technology is incorporated into the sensor suite to provide additional arousal level detection. For example, several currently-available smart watches use photoplethysmography (PPG) to measure or estimate blood pressure and/or heart rate based on a measured among of light reflected from skin to quantitatively capture blood flow in the peripheral vasculature based on the light absorption properties of hemoglobin. Such a smart watch is a useful sensor for the Phase 1 design, however, it requires compliance of the user, and the monitoring could be disabled by simply non wearing the smart watch while accessing problematic content on the internet, or by having another person wear the smart watch while the intended user accesses problematic content on the internet. Standard PPG technology requires contact with the skin of the user to perform its measurements.

Research is now extending PPG to remote, camera-based PPG (rPPG) which does not require physical contact with the user's skin. For example, Curran, et al., described in “Camera-based remote photoplethysmography for blood pressure measurement: current evidence, clinical perspectives, and future applications” (Conn. Health Teleme.d, 2023) how the proliferation of very high-resolution cameras with excellent lens capabilities is allowing smart phone cameras to implement rPPG by imaging a visible part of the user user's body or face, such as their cheeks or forehead.

Through the advanced use of such affordable and widely-used cameras on smart phones, a user's respiratory rate (RR), heart rate (HR), blood oxygen saturation (SpO2), and even atrial fibrillation may be detected. For example, Curran, et al., provide this explanation of how it does this:
“Whether using a contact sensor or a camera, the process of estimating BP from reflectance mode PPG shares similarities . . . . First, a light source is needed, either from a device or ambient light. Generally, the visible spectrum of light is used, though infrared has been explored as it offers deeper penetration into dermal layers containing arteries . . . . Light is shone on the skin and absorbed in part by blood. The green channel is conventionally used to isolate hemoglobin, the major component of blood, based on its absorption properties. Pulsatile blood flow within arterioles and the microvascular bed in the skin results in fluctuating levels of absorption by hemoglobin. For rPPG, a region of interest (ROI) is selected, commonly the forehead or cheeks, due to the larger surface area and/or perpendicular position to the lens when facing he camera. The reflected light is returned to a photodetector and visualized as a waveform that resembles an attenuated arterial pressure tracing. Next, the PPG signal quality is improved by spatial averaging, normalization, low and high pass filtering, and other post-processing methods to remove noise from motion artifacts or non-hemoglobin components such as melanin. The first and second derivatives of the waveform may also be computed. Finally, the cleaned PPG signal is fit to a predetermined parametric model to produce an estimate of BP based on waveform characteristics, patient demographics, or other parameters.” (Curran, et al.)

In this manner, additional arousal detection can be made by embodiments of the present invention using rPPG via a web-connected device's built-in camera, via one or more cameras added to a user's web-connected device, and/or via one or more cameras in a vicinity of the user, such as security cameras in a library, school room, mental health facility, jail, prison, or vehicle, which have the region of interest of the user in its or their view.

Clinical Trial Data. During clinical trials of one or more embodiments of the present invention, one or more therapists will use one or more instruments to measure executive functions for each of the three ranges of arousal (low, safe range and high), including but not limited to the Stroop Test, Digit Span Quick Test (DPQT) and an ANTI-Vea Test. Telemetry ranges and the instrument scores will be recorded and compiled into a bell curve. This data may become a baseline reference which may be used on a server or a client device application program, such that these research statistics can be loaded into a device dashboard and selectively and independently pulled.

Additional Research Information for Use. Research by Zhu, et al., which described in “Molecular and Functional Imaging of Internet Addiction” in BioMed Research International (2015) showed that excessive internet use exhibits biological changes in the user's brain's prefrontal cortex that mirror changes seen in other addiction syndromes. Neuroimaging showed changes in brain structure occurring in the temporal cortex and ventral striatum associated with compromised executive function and increased impulse risk occurs with uncontrolled internet use. The physiological changes affect the brain as evidenced in Hilton's 2011 study that found addiction disorders are labeled by neurologists as hypofrontal syndromes resulting in damage to the “breaking system” of the brain. They label two types of addiction disorders, endogenous where the user manipulates the dopamine production of the body through stimulus and exogenous where the user manipulates dopamine production using a substance. Research in 2015 shows that excessive internet use exhibits biological changes in the prefrontal cortex that mirror changes seen in other addiction syndromes. Neuroimaging has shown changes in brain structure in the temporal cortex and ventral striatum associated with compromised executive function and increased impulse risk occurs with uncontrolled internet use (Zhu, Yunqi & Zhang, Hong & Tian, Mei).

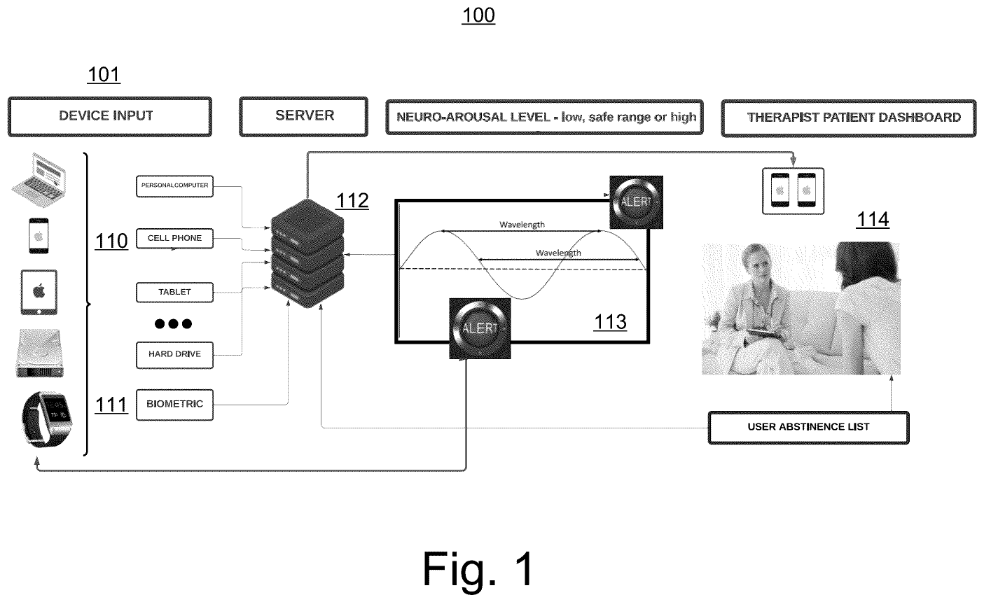

Platform Architecture. Referring to FIG. 1, an architecture for a platform 100 according to at least one embodiment of the present invention is shown. One or more user devices 101, including user devices 110 for accessing internet content such as but not limited to personal computers, tablet computers, smart phones, smart watches, gaming consoles, removable data drives, static data drives, etc., and one or more biometric sensors 111, such as but not limited to photoplethysmography (PPG), remote photoplethysmography (rPPG), respiratory rate (RR), heart rate (HR), blood oxygen saturation (SpO2), blood pressure (BP), and galvanic skin response (GSR), provide data regarding user biometric status and user interaction activity with internet content to one or more servers 101. The user's arousal level is determine based on one or more factors including analysis of the biometric sensor data, analysis of the context of the interaction activities, and optionally user-specific profile settings.

The determined user arousal level is compared to one or more rules regarding arousal threshold declaration, optionally keyed to interaction activity and/or content type, to declare the user's arousal state, such as but not limited to low, safe, or high. If a high arousal declaration is made, the user's current interaction activity is recorded into a secure digital log, which is then accessible by a user interface dashboard 114 by a therapist for use during a counselling session. Optionally, one or more real-time alerts may also be sent to one or more administrative consoles, such as a health professional (e.g., a psychiatric nurse, etc.) at a mental health facility, a guard at a detention facility, a librarian at a library, etc.

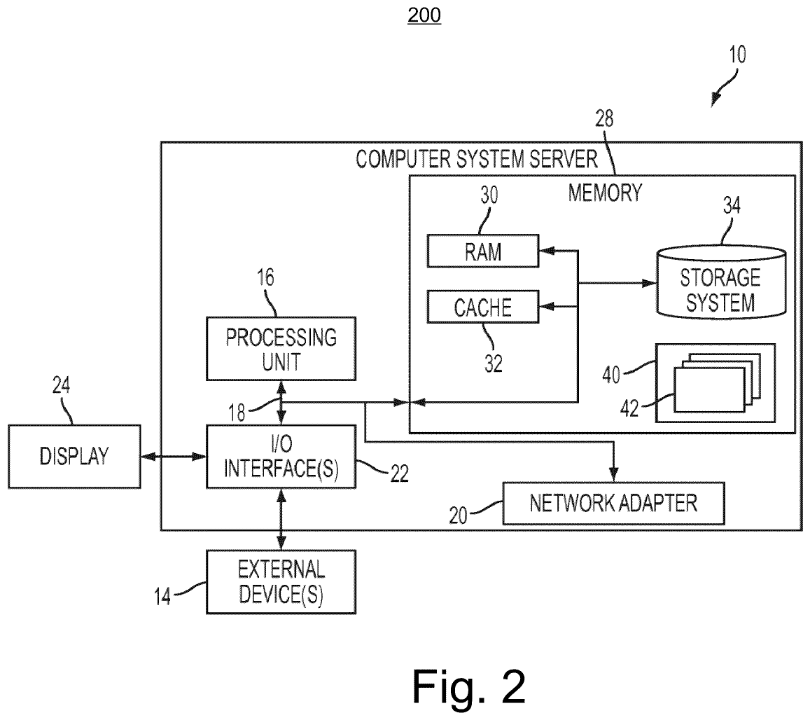

Computing Platforms. Referring now to FIG. 2, a schematic of an example of a computer system/server 10 is shown suitable for embodiment of the present invention. The present invention may be realized as certain improvements to the functioning of basic computing platforms by addition of one or more of application programs, operating system extensions, circuit cards, memory, communication interfaces, long-term data storage, and various user interface devices, such as those previously mentioned herein.

The suitable computer system/server 10 is shown in in a functional organization manner, capable of performing one or more computer-implemented processes to achieve the operational improvements described in the foregoing paragraphs. The components of the computer system/server 10 may include, but are not limited to, one or more processors or processing units 16, a system memory 28, and a bus 18 that couples various system components including the system memory 28 to the processor 16. The computer system/server 10 may perform functions as different machine types depending on the role in the system the function is related to. For example, depending on the function being implemented at any given time when interfacing with the system, the computer system/server 10 may be for example, personal computer systems, tablet devices, mobile telephone devices, server computer systems, handheld or laptop devices, multiprocessor systems, microprocessor-based systems, set top boxes, programmable consumer electronics, network PCs, and distributed cloud computing environments that include any of the above systems or devices, and the like. The computer system/server 10 may be described in the general context of computer system executable instructions, such as program modules, being executed by a computer system (described for example, below). In some embodiments, the computer system/server 10 may be a cloud computing node connected to a cloud computing network (not shown). Is that OK to say. The computer system/server 10 may be practiced in distributed cloud computing environments where tasks described above are performed by remote processing devices that are linked through a communications network. In a distributed cloud computing environment, program modules may be located in both local and remote computer system storage media including memory storage devices.

The improved computer system/server 10 may include a variety of computer system readable media. Such media could be chosen from any available media that is accessible by the computer system/server 10, including non-transitory, volatile and non-volatile media, removable and non-removable media. The system memory 28 could include one or more computer system readable media in the form of volatile memory, such as a random access memory (RAM) 30 and/or a cache memory 32. By way of example only, a storage system 34 can be provided for reading from and writing to a non-removable, non-volatile magnetic media device. The system memory 28 may include at least one program product 40 having a set (e.g., at least one) of program modules 42 that are configured to carry out the functions of embodiments of the invention. The program product/utility 40, having a set (at least one) of program modules 42, may be stored in the system memory 28 by way of example, and not limitation, as well as an operating system, one or more application programs, other program modules, and program data. Each of the operating system, one or more application programs, other program modules, and program data or some combination thereof, may include an implementation of a networking environment. The program modules 42 generally carry out the functions and/or methodologies of keystroke logging and identification of prohibited behavior in online cyberactivity embodiments as described above with respect to FIG. 1.

The computer system/server 10 may also communicate with one or more external devices 14 such as a keyboard, a pointing device, a display 24, etc.; and/or any devices (e.g., network card, modem, etc.) that enable the computer system/server 10 to communicate with one or more other computing devices. Such communication can occur via Input/Output (I/O) interfaces 22. Alternatively, the computer system/server 10 can communicate with one or more networks such as a local area network (LAN), a general wide area network (WAN), and/or a public network (e.g., the Internet) via a network adapter 20 (for example, through a network as shown in FIG. 2). As depicted, the network adapter 20 may communicate with the other components of the computer system/server 10 via the bus 18.

As will be appreciated by one skilled in the art, aspects of the disclosed invention may be embodied as a system, method or process, or computer program product. Accordingly, aspects of the disclosed invention may take the form of an entirely hardware embodiment, an entirely software embodiment (including firmware, resident software, microcode, etc.) or an embodiment combining software and hardware aspects that may all generally be referred to herein as a “circuit,” “module,” or “system.” Furthermore, aspects of the disclosed invention may take the form of a computer program product embodied in one or more computer readable media having computer readable program code embodied thereon.

Any combination of one or more computer readable media (for example, storage system 34) may be utilized. In the context of this disclosure, a computer readable storage medium may be any tangible or non-transitory medium that can contain, or store a program (for example, the program product 40) for use by or in connection with an instruction execution system, apparatus, or device. A computer readable storage medium may be, for example, but not limited to, an electronic, magnetic, optical, electromagnetic, infrared, or semiconductor system, apparatus, or device, or any suitable combination of the foregoing.

Aspects of the disclosed invention are described below with reference to block diagrams of methods, apparatus (systems) and computer program products according to embodiments of the invention. It will be understood that each block of the block diagrams, and combinations of blocks in the flowchart illustrations and/or block diagrams, can be implemented by computer program instructions. These computer program instructions may be provided to the processor 16 of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

I/We claim:

1. A system for online behavior modification of a user comprising:
at least one internet-connected device which accesses internet content under the control of a user;
at least one content monitor which records, into a computer-readable memory structure in a computer-readable memory device, one or more identifiers of internet content accessed by the internet-connected device under the control of the user;
at least one biometric sensor sensing one or more biometric conditions of the user concurrently with the accessing of the internet content;
an arousal detection filter which declares a level of arousal of the user associated with accessing internet content identified by the identifiers and the one or more biometric conditions;
an activity logger recording, into a user activity log stored in computer-readable memory device, the declared level of arousal and the identifiers; and
a server computer system receiving the one or more identifiers and the one or more biometric conditions associated with the user and the access.

2. The system as set forth in claim 1 further comprising a dashboard portion of a computer program executed by a computer, retrieving the activity log and displaying one or more records in a user-readable format on a computer user interface of the computer.

3. The system as set forth in claim 2 wherein the dashboard further receives from an administrator user one or more inputs to review, modify and save one or more user-specific criteria, and wherein the arousal detection filter further uses the user-specific criteria in the declaring of the level of arousal of the user.

4. The system as set forth in claim 1 wherein the at least one internet-connected device comprises one or more devices selected from the group consisting of a smart watch, a smart phone, a game system, a tablet computer, and a personal computer.

5. The system as set forth in claim 1 wherein the at least one biometric sensor comprises one or more sensors selected from the group consisting of a photoplethysmography (PPG) sensor, a remote photoplethysmography (rPPG) sensor, a respiratory rate (RR) sensor, a heart rate (HR) sensor, a blood oxygen saturation (SpO2) sensor, a blood pressure (BP) sensor, and a galvanic skin response (GSR) sensor.

6. The system as set forth in claim 1 wherein the one or more identifiers of internet content comprises an identifier selected from the group consisting of a web page type, a streaming audio type, a streaming video type, a movie rating type, a Universal Resource Locator (URL), an internet protocol (IP) address, a removable memory type, a non-removable memory type, a data connection encryption type, and a virtual private network (VPN) type.

7. The system as set forth in claim 1 wherein the user activity log is stored in a database computer-readable memory structure.

8. The system as set forth in claim 1 wherein the user activity log is stored in an encrypted computer-readable memory structure.

9. A computer program product for assisting and facilitating online behavior modification of a user, the computer program product comprising:
one or more computer-readable memory devices which is not a propagating signal per se;
one or more computer program instructions stored by the one or more computer-readable memory devices which, when executed by one or more computer processors, cause one or more computer processors to perform steps comprising:
accessing, under user control, internet content via an internet-connected device;
recording by at least one content monitor into a computer-readable memory structure in a computer-readable memory device, one or more identifiers of internet content accessed by the internet-connected device under the control of the user;
receiving from at least one biometric sensor one or more biometric conditions of the user concurrently with the accessing of the internet content;
declaring by an arousal detection filter a level of arousal of the user associated with accessing internet content identified by the identifiers and the one or more biometric conditions;
logging user activity, into an activity log stored in computer-readable memory device, the declared level of arousal and the identifiers; and
receiving, by a server process on a server computer system, the one or more identifiers and the one or more biometric conditions associated with the user and the access.

10. The computer program product as set forth in claim 9 wherein the one or more computer program instructions further comprise instructions for providing a dashboard which retrieves the activity log and displays one or more records in a user-readable format on a computer user interface of the computer.

11. The computer program product as set forth in claim 10 wherein the dashboard further receives from an administrator user one or more inputs to review, modify and save one or more user-specific criteria, and wherein the arousal detection filter further uses the user-specific criteria in the declaring of the level of arousal of the user.

12. The computer program product as set forth in claim 9 wherein the at least one internet-connected device comprises one or more devices selected from the group consisting of a smart watch, a smart phone, a game system, a tablet computer, and a personal computer.

13. The computer program product as set forth in claim 9 wherein the at least one biometric sensor comprises one or more sensors selected from the group consisting of a photoplethysmography (PPG) sensor, a remote photoplethysmography (rPPG) sensor, a respiratory rate (RR) sensor, a heart rate (HR) sensor, a blood oxygen saturation (SpO2) sensor, a blood pressure (BP) sensor, and a galvanic skin response (GSR) sensor.

14. The computer program product as set forth in claim 9 wherein the one or more identifiers of internet content comprises an identifier selected from the group consisting of a web page type, a streaming audio type, a streaming video type, a movie rating type, a Universal Resource Locator (URL), an internet protocol (IP) address, a removable memory type, a non-removable memory type, a data connection encryption type, and a virtual private network (VPN) type.

15. The computer program product as set forth in claim 9 wherein the user activity log is stored in a database computer-readable memory structure.

16. The computer program product as set forth in claim 9 wherein the user activity log is stored in an encrypted computer-readable memory structure.

17. A method for assisting and facilitating online behavior modification of a user, the method comprising:
accessing, under user control, internet content via an internet-connected device;
automatically recording, by at least one content monitor, into a computer-readable memory structure in a computer-readable memory device, one or more identifiers of internet content accessed by the internet-connected device under the control of the user;
automatically receiving, from at least one biometric sensor, one or more biometric conditions of the user concurrently with the accessing of the internet content;
automatically declaring, by an arousal detection filter, a level of arousal of the user associated with accessing internet content identified by the identifiers and the one or more biometric conditions;
automatically logging user activity into an activity log stored in computer-readable memory device, the declared level of arousal and the identifiers; and
automatically sending to a server computer system the one or more identifiers and the one or more biometric conditions associated with the user and the access.

18. The method as set forth in claim 17 wherein the at least one internet-connected device comprises one or more devices selected from the group consisting of a smart watch, a smart phone, a game system, a tablet computer, and a personal computer, wherein the at least one biometric sensor comprises one or more sensors selected from the group consisting of a photoplethysmography (PPG) sensor, a remote photoplethysmography (rPPG) sensor, a respiratory rate (RR) sensor, a heart rate (HR) sensor, a blood oxygen saturation (SpO2) sensor, a blood pressure (BP) sensor, and a galvanic skin response (GSR) sensor; and wherein the one or more identifiers of internet content comprises an identifier selected from the group consisting of a web page type, a streaming audio type, a streaming video type, a movie rating type, a Universal Resource Locator (URL), an internet protocol (IP) address, a removable memory type, a non-removable memory type, a data connection encryption type, and a virtual private network (VPN) type.

19. The method as set forth in claim 17 wherein the user activity log is stored in a database computer-readable memory structure.

20. The method as set forth in claim 17 wherein the user activity log is stored in an encrypted computer-readable memory structure.
